(19) United States
(12) Patent Application Publication
(10) Pub. No.: US 2023/0051204 A1
(43) Pub. Date: Feb. 16, 2023

(54) ANTIMICROBIAL COMPOSITION CONTAINING WASP EXTRACT AS ACTIVE INGREDIENT

(71) Applicant: Andong National University Industry-Academic cooperation Foundation, Gyeongsangbuk-do (KR)

(72) Inventor: Ho Yong SOHN, Daegu (KR)

(21) Appl. No.: 17/553,517

(22) Filed: Dec. 16, 2021

(30) Foreign Application Priority Data
Jul. 27, 2021 (KR) 10-2021-0098695

(51) Int. Cl. A61K 35/64; C11D 3/48; C11D 3/38; A23L 33/10; A23K 10/20

(52) U.S. Cl. CPC A61K 35/64; C11D 3/48; C11D 3/38; A23L 33/10; A23K 10/20; A23V 2002/00

(57) ABSTRACT

An antimicrobial composition containing a wasp extract, and more particularly, a broad-spectrum antimicrobial composition containing, as an active ingredient, an ethyl acetate fraction recovered by sequential organic solvent fraction of an ethanol extract of Vespa velutina nigrithorax or Vespa simillima simillima Smith. The Vespa velutina nigrithorax extract or the V. simillima simillima Smith extract exhibits excellent broad-spectrum antimicrobial activity against gram-positive bacteria, gram-negative bacterial and fungi, and thus may be used for pharmaceutical compositions, health functional foods, cosmetic compositions, animal feed compositions, additives for food, cosmetics or animal feed, various detergents and various cleansers. In addition, the extract has excellent heat stability, and does not lose its activity even under an acidic condition of pH 2.

### BACKGROUND

The present disclosure relates to an antimicrobial composition containing a wasp extract, and more particularly, to a broad-spectrum antimicrobial composition containing, as an active ingredient, an ethyl acetate fraction recovered by sequential organic solvent fraction of an ethanol extract of Vespa velutina nigrithorax or Vespa simillima simillima Smith.

Humans have been using various antimicrobial agents and preservatives to defend against the invasion of pathogenic microorganisms and to control food spoilage microorganisms. However, chemical antimicrobial agents and preservatives exhibit excellent antimicrobial activity against various bacteria and fungi, but when they are used for a long time, they show many side effects such as allergic redness, angioedema, bone marrow suppression, anemia, gastritis, and degenerative brain disease. In fact, in the United States, the use of antibiotics with severe side effects, such as chloramphenicol, has been banned. Therefore, various studies have been conducted on the development of natural antimicrobial agents using natural products that have been used for a long time while ensuring safety.

Meanwhile, according to the U.S. Center for Disease Control and Prevention, in-hospital bloodstream infection is one of the leading causes of death in the United States. In addition, infection with pathogenic bacteria and fungi through the skin and soft tissue is recognized as a universal medical disease, and is understood to be caused mainly by trauma or surgery.

Many pathogenic bacterial and fungal infections have different treatment methods depending on the severity of the infection, but can be successfully treated by oral antimicrobial agents and topical cleansing. However, more severe or complex infections that commonly occur in patients with underlying risk factors (e.g., vascular blood flow abnormalities, diabetes, etc.) and/or infections caused by difficult-to-treat or multi-resistant bacteria and fungi may require measures such as intravenous antimicrobial therapy and invasive surgical tissue removal. For example, vancomycin, a glycopeptide antibiotic, has been successfully used against severe nosocomial infections caused by multi-drug-resistant Gram-positive bacteria, particularly MRSA, coagulase-negative Staphylococcus and Enterococcus. However, it has been recently reported that the isolation of strains resistant to vancomycin is increasing. In addition, it is true that the treatment of severe infections, caused by some pathogens, with the current antibiotics, is very limited. Therefore, there is an increasing need for the development of more effective new antibiotics against increasingly resistant bacteria.

Meanwhile, wasps are insects belonging to the family Vespidae of the order Hymenopterathe, and are predatory insects that eat bees and other small insects. There are about 30 wasp species of 5 genera (Vespa, Vespula, Dolichovespula, Parapolybia, and Polistes) worldwide. In Korea, the following 10 wasp species are known: Vespa analis parallela Andre, V. baslis Smith, V. ducalis Smith, V. dybowskii Andre, Vespa crabro crabroniformis Smith, V. crabro flavofasciata Cameron, V. mandarinia Smith, V. simillima simillima Smith, V. smillima xanthoptera Cameron, and V. velutina nigrithorax. Wasps have different habitats and distributions depending on the species, build wasp nests and inhabit in groups mainly in low mountains, treetops or in the ground.

Wasps are also responsible for pollination and perform the roles of parasites and predators. In particular, some wasps are recognized as pests in the beekeeping industry because they prey on adult honeybees, and damage by them is gradually increasing. In recent years, wasps have been a threat to humans and animals due to wasp stings, and multiple stings with the powerful wasp venom can sometimes cause death. Thus, in order to prevent damage caused by wasps, studies on wasp attraction and capture (Jung Jun-Seong et al., 2018. Trends in Agriculture & Life Sciences 56: 35-45) and the control of wasps using a specific frequency (Kim et al., 2019. J. Apiculture 34: 7-13) have been conducted.

In Korea, wasps have been recognized as a nourishing tonic food since ancient times, and as a representative example, wasp wine made by soaking wasps in alcohol has been used for the treatment of inflammation, epilepsy, convulsions and dental diseases (Heo Jun's Donguibogam, 1615). However, studies on wasps are very limited, and most of the existing studies are studies on ecology and classification of wasps (Murat et al., 2016, Carbohydrate Polymers 145: 64-70) and wasp venom (Xinwang et al., 2013, Toxicon 74: 151-157: Yoon et al., 2015, Journal of Asia-Pacific Entomology 18: 815-823), and there are no studies on the useful physiological activity of wasps themselves. A study on wasp venom has reported the strong hemolytic activity and cytotoxicity of the wasp venom, and recently, anti-inflammatory activity, allergy induction and neurotoxicity by histamine, serotonin, phospholipase A2, hyaluronidase, etc. have been found (Lee, B. H., Park, H. J. 1998. J. Korean Environ. Sci. Sco. 62-66; Sabe et al., 2017. Med. Inflamm. 6978194. doi: 10.1155/2017/6978194). In addition, damage caused by acute thrombosis after a wasp sting has been continuously reported (Chen, D M. et al., 2004. The American journal of medicine 116: 567-569; Min J H et al., 2013. Korean circulation journal 43: 561-564).

An antimicrobial peptide (e.g., mastoparan) contained in wasp venom is known to be difficult to use as an actual antibacterial agent due to its strong red blood cell hemolytic activity (Ha Yeon-Jo, 2019 Gyeongsang National University doctoral thesis). However, until now, there has been no report on an ethyl acetate fraction of an extract of Vespa velutina nigrithorax or V. simillima simillima Smith, which exhibits strong broad-spectrum antimicrobial activity without showing human erythrocyte hemolytic activity.

Patents related to wasps include Korean Patent No. 10-2028363 [entitled “System for combating harmful wasps”] and Korean Patent No. 10-2075057 [entitled “Wasp trapping device capable of collecting wasps alive”], Korean Patent No. 10-1972070 [entitled “Composition for preventing or treating gout containing bee venom isolated from worker bees of V. mandarinia Smith”], Korean Patent No. 10-1972074 [entitled “Composition for preventing or treating Alzheimer's disease containing bee venom isolated from worker bees of V. mandarinia Smith”], Korean Patent No. 10-1374327 [entitled “Functional cosmetic composition containing V. mandarinia Smith venom extract”], Korean Patent Application Publication No. 10-1999-0039050 [entitled “Agent for preventing and treating acne containing wasp venom extract”], Korean Patent Application Publication No. 10-2012-0111206 [entitled “Method of preparing functional cosmetic composition using wasps”], Korean Patent Application Publication No. 10-2012-0100450 [entitled “Method of preparing functional cosmetic composition using V. mandarinia Smith], and Korean Patent Application Publication No. 10-2005-0028992 [“Method of producing traditional wine using wasps and traditional wine liquor using wasps produced thereby”]. However, until now, there has been no known patent related to the strong broad-spectrum antimicrobial activity of an ethyl acetate fraction of an extract of Vespa velutina nigrithorax or V. simillima simillima Smith.
Prior Art Document: Ha Yeon-Jo, 2019 Gyeongsang National University doctoral thesis

### SUMMARY OF THE INVENTION

The present disclosure has been made in order to solve the above-described problems occurring in the prior art, and an object of the present disclosure is to provide an antimicrobial composition containing, as an active ingredient, a wasp extract selected from the group consisting of a Vespa velutina nigrithorax extract and a V. simillima simillima Smith extract.

To achieve the above object, the present disclosure provides an antimicrobial composition containing, as an active ingredient, a wasp extract selected from the group consisting of a Vespa velutina nigrithorax extract and a V. simillima simillima Smith extract.

The extract is preferably an ethanol extract.

The extract is preferably an ethyl acetate fraction obtained after ethanol extraction.

The antimicrobial composition is preferably selected from the group consisting of pharmaceutical compositions, health functional foods, cosmetic compositions, animal feed compositions, food additives, cosmetic additives, animal feed additives, detergents, and cleansers.

### BRIEF DESCRIPTION OF THE DRAWINGS

FIGURE shows photographs of V. mandarinia Smith, V. simillima simillima Smith and Vespa velutina nigrithorax used in the Examples of the present disclosure.

### DETAILED DESCRIPTION OF THE INVENTION

Hereinafter, the present disclosure will be described in detail.

In order to test the antithrombotic efficacy of wasps, the present inventors first collected V. mandarinia Smith, V. simillima simillima Smith and Vespa velutina nigrithorax in Korea, and then washed the collected wasps with water to remove foreign substances, followed by soaking in 30% ethanol and 1 year of leaching. Thereafter, the extracts were filtered and concentrated under reduced pressure to obtain ethanol extracts. From each of the extracts, a hexene fraction, an ethyl acetate fraction, a butanol fraction and water residue, which are sequential organic fractions, were recovered. The ethyl acetate fraction of the V. simillima simillima Smith extract or the Vespa velutina nigrithorax extract was identified to be antimicrobial active ingredient, and it was confirmed that the active ingredient is characterized by having excellent heat stability and acid stability. Thus, the ethyl acetate fraction of the V. simillima simillima Smith extract or the Vespa velutina nigrithorax extract was intended to be used as an active ingredient for an antimicrobial composition.

Therefore, the present disclosure provides an antimicrobial composition containing, as an active ingredient, a wasp extract selected from the group consisting of a Vespa velutina nigrithorax extract or a V. simillima simillima Smith extract.

The extract is preferably an ethanol extract.

The extract is preferably an ethyl acetate fraction obtained after ethanol extraction.

The antimicrobial composition is preferably selected from the group consisting of pharmaceutical compositions, health functional foods, cosmetic compositions, animal feed compositions, food additives, cosmetic additives, animal feed additives, detergents, and cleansers.

Hereinafter, a method for preparing an ethyl acetate fraction of a Vespa velutina nigrithorax extract or V. simillima simillima Smith according to the present disclosure and a test for the efficacy of the ethyl acetate fraction will be described in more detail.

In order to achieve the object of the present disclosure, the present inventors performed an experimental method including steps of: collecting three wasp species; preparing wasp extracts; preparing sequential organic solvent fractions (hexene, ethyl acetate and butanol fractions) from each of the wasp extracts and then preparing water residue; evaluating the antimicrobial activities of the extracts and the fractions; and examining the stability of each ethyl acetate fraction as an active ingredient.

The wasp extract contained in the composition of the present disclosure may be obtained by a method including steps of: extracting wasps with 30% ethanol; and filtering the extract through a filter having a mesh size of 0.06 mm or less and concentrating the filtrate under reduced pressure.

A solvent that is used in the present disclosure may be water (cold or hot water), spirit, an anhydrous or hydrous lower alcohol having 1 to 4 carbon atoms (e.g., methanol, ethanol, spirit, propanol, butanol, etc.), or a mixed solvent of the lower alcohol and water, and hot-water extraction or ethanol extraction is most preferred.

In a preferred embodiment of the present disclosure, wasps may be extracted with hot water or ethanol. In addition, the hot-water or ethanol extract may be fractionated sequentially with organic solvents (hexene, ethyl acetate and butanol) or fractioned with each of the organic solvents to additionally obtain a hexene fraction, an ethyl acetate fraction, a butanol fraction and water residue.

The wasp extract of the present disclosure may be prepared into powder through a conventional powdering process such as vacuum drying, freeze-drying, or spray-drying. The extract is not degraded by various degrading enzymes in plasma, and remains active even upon heat treatment at 100° C. and in the human stomach at pH 2.

In a preferred embodiment, the antimicrobial composition of the present disclosure may be applied as a pharmaceutical composition.

For use, the pharmaceutical composition containing the active ingredient of the present disclosure may be formulated in various forms, including oral dosage forms such as powders, granules, tablets, capsules, suspensions, emulsions, syrups, and aerosols, or injection dosage forms sterile injections, according to conventional methods depending on the intended use thereof, and may be administered through various routes including oral, intravenous, intraperitoneal, subcutaneous, rectal and topical routes. This pharmaceutical composition may further contain a carrier, excipient or diluent. Examples of a carrier, excipient or diluent that may be contained in the pharmaceutical composition include lactose, dextrose, sucrose, sorbitol, mannitol, xylitol, erythritol, maltitol, starch, gum acacia, alginate, gelatin, calcium phosphate, calcium silicate, cellulose, methyl cellulose, amorphous cellulose, polyvinyl pyrrolidone, water, methylhydroxybenzoate, propylhydroxybenzoate, talc, magnesium stearate, and mineral oil. In addition, the pharmaceutical composition of the present disclosure may further contain a filler, an anti-aggregating agent, a lubricant, a wetting agent, a flavoring agent, an emulsifying agent, a preservative, and the like.

The pharmaceutical composition of the present disclosure is administered in a pharmaceutically effective amount. As used herein, the term “pharmaceutically effective amount” refers to an amount sufficient to treat a disease at a reasonable benefit/risk ratio applicable to any medical treatment. The effective dose level of the active ingredient may be determined depending on factors, including the kind and severity of the patient's disease, the activity of the drug, sensitivity to the drug, the time of administration, the route of administration, excretion rate, the duration of treatment, and drugs used in combination with the composition, as well as other factors well known in the medical field. The pharmaceutical composition of the present disclosure may be administered individually or in combination with other therapeutic agents, and may be administered sequentially or simultaneously with conventional therapeutic agents. The pharmaceutical composition may be administered in a single or multiple dosage form. It is important to administer the pharmaceutical composition in the minimum amount that can exhibit the maximum effect without causing side effects, in view of all the above-described factors, and this amount can be easily determined by a person skilled in the art.

The effective amount of the active ingredient having antimicrobial activity in the pharmaceutical composition of the present disclosure may vary depending on the patient's age, sex and body weight. Generally, the active ingredient may be administered daily or every other day at a dose of 1 to 5,000 mg/kg body weight, preferably 100 to 3,000 mg/kg body weight, or may be administered 1 to 3 times a day at this dose. However, since the dose may increase or decrease depending on the route of administration, the severity of the disease, the patient's sex, weight and age, etc., the dose is not intended to limit the scope of the present disclosure in any way. The pharmaceutical composition of the present disclosure may be administered to a subject through various routes. All modes of administration can be contemplated, and for example, the pharmaceutical composition may be administered orally, intrarectally, or by intrarectal, intravenous, intramuscular, subcutaneous, intrauterine, intrathecal or intra-cerebroventricular injection. In the present disclosure, the term “administration” means providing a given substance to a patient by any suitable method. The pharmaceutical composition of the present disclosure may be administered orally or parenterally through all general routes as long as it can reach the target tissue. In addition, the composition of the present disclosure may also be administered using any device capable of delivering the active ingredient to target cells. In the present disclosure, the term “subject” is not particularly limited, but includes, for example, humans, monkeys, cattle, horses, sheep, pigs, chicken, turkeys, quails, cats, dogs, mice, rats, rabbits or guinea pigs, and preferably refers to mammals, more preferably humans.

In a preferred embodiment, the antimicrobial composition of the present disclosure may be applied as a health functional food.

The health functional food of the present disclosure may be variously used in effective foods and beverages that can lower the possibility of infection in a subject with a high risk of infection by pathogenic microorganisms. Foods containing the active ingredient having excellent antimicrobial activity according to the present disclosure include various foods, for example, beverages, gums, teas, vitamin complexes, health supplement foods and the like, and may be used in the form of powders, granules, tablets, capsules or beverages. The active ingredient of the present disclosure may generally be added in an amount of 0.01 to 15 wt % based on the total food weight. For a health beverage composition, the active ingredient may be added in an amount of 0.02 to 10 g, preferably 0.3 to 1 g, based on 100 ml of the health beverage composition. The health functional food of the present disclosure may additionally contain food-acceptable additives, for example, natural carbohydrates and various flavoring agents, in addition to containing the wasp extract as an essential component at the indicated percentage. Examples of the natural carbohydrates include conventional sugars, such as monosaccharides (e.g., glucose, fructose, etc.), disaccharides (e.g., maltose, sucrose, etc.), polysaccharides (e.g., dextrin, cyclodextrin, etc.), and sugar alcohols such as xylitol, sorbitol, erythritol or the like. Examples of the flavoring agents that may be used in the present disclosure include thaumatin, rebaudioside A, glycyrrhizin, saccharin, aspartame, etc. The flavoring agent is used in an amount of about 1 to 20 g, preferably about 5 to 12 g, based on 100 mL of the health functional food of the present disclosure. In addition, the health functional food of the present disclosure may contain various nutrients, vitamins, minerals, flavoring agents such as synthetic flavoring agents and natural flavoring agents, colorants, extenders, pectic acid and its salt, alginic acid and its salt, organic acids, protective colloidal thickeners, pH adjusting agents, stabilizers, preservatives, glycerin, alcohol, carbonizing agents that are used in carbonated beverages, etc. Additionally, the health functional food of the present disclosure may contain fruit flesh that is used for the preparation of natural fruit juice, fruit juice beverages or vegetable beverages. These components may be used individually or in combination. The content of these additives is generally selected in the range of 0.01 to about 20 parts by weight based on 100 parts by weight of the health functional food.

In a preferred embodiment, the antimicrobial composition of the present disclosure may be applied as a cosmetic composition.

The cosmetic composition containing the active ingredient having excellent antimicrobial activity according to the present disclosure may be prepared in the form of, for example, essence, nourishing cream, body lotion, rinse, shampoo, body cleanser, lotion, skin lotion, mask pack, etc., but is not limited thereto. Preparation of the cosmetic composition in the above form may be easily carried out according to various manufacturing processes known to those skilled in the art.

In a preferred embodiment, the antimicrobial composition of the present disclosure may be applied as an animal feed composition.

The term “animal” refers to a group of non-plant organisms which mainly ingest organic matter as nutrients and in which digestive, excretory and respiratory organs are differentiated. Specific examples of the animal include echinoderms, crustaceans, molluscs, fish, amphibians, reptiles, birds, and mammals. Preferred examples of the animal include echinoderms such as sea urchins or sea cucumbers, arthropods including crustaceans such as crabs, shrimps and lobsters, molluscs such as cephalopods, gastropods or bivalves, fish such as red sea bream, sea bream, cod, flounder or halibut, birds including poultry such as pheasants or chickens, or mammals such as pigs, cattle and goats. The animal feed composition may further contain grains, vegetable protein feed, animal protein feed, sugar or dairy products, in addition to the antimicrobial composition containing the antimicrobial ingredient of the present disclosure. Specific examples of the grains include pulverized or crushed wheat, oats, barley, corn and rice, and specific examples of the vegetable protein feed include those containing rapeseed, soybean and sunflower seed as main components. Specific examples of the animal protein feed include blood meal, meat meal, bone meal and fish meal, and specific examples of the sugar or dairy products include dry components consisting of various milk powder and whey powder.

The animal feed composition may additionally contain ingredients such as nutritional supplements, digestion and absorption enhancers, growth promoters or disease prevention agents.

The content of the antimicrobial composition in the animal feed composition of the present disclosure may vary depending on the purpose and conditions of use of the feed, and for example, the antimicrobial composition may be contained in an amount of 0.1 g to 100 g based on 1 kg of the finally produced feed.

In addition, the feed composition may be prepared as a consistent granulated or granular material depending on the degree of pulverization of the components. The composition may be fed through a mesh or formed into a desired discrete shape for further processing and packaging, and may be subjected to pelletization, expansion or extrusion processes for storage. For ease of storage, excess water may preferably be dried off from the composition.

In a preferred embodiment, the antimicrobial composition of the present disclosure may be applied as a food additive, a cosmetic product or animal feed.

The additive is added to impart storage stability to food, cosmetics or animal feed used, and the antimicrobial composition that is a natural ingredient is not harmful to the human body, and thus may be safely applied to food, cosmetics or animal feed.

The additive may further include additional other additives appropriately selected according to the intended use, and may be used in combination with other active ingredients or commonly used additives such as pigments, surfactants or preservatives.

The additive may be produced as powder, granules, tablets or liquid depending on the intended use thereof, and a conventional method may be used for packaging for commercialization thereof.

In addition, the content of the antimicrobial composition in the additive may be appropriately selected depending on the intended use, application form, application site and application target article thereof, and may be, for example, 0.01 parts by weight to 50 parts by weight based on the total weight of the additive composition, but the content of the antimicrobial composition is not limited thereto. Since the antimicrobial composition of the present invention is a natural component that is not harmful to the human body, it may be used in various amounts as long as it can achieve the application purpose due to the nature of the product.

In a preferred embodiment, the antimicrobial composition of the present disclosure may be applied as a detergent. The detergent may be preferably a detergent for clothes, a dishwashing agent, a food cleaner, and a household appliance cleaner. Since the antibacterial composition is a natural component that is not harmful to the human body, it can more preferably be used for the purpose of washing various household items including kitchenware or food washing or for the purpose of imparting antibacterial properties.

The detergent may further contain an additive selected appropriately depending on the intended use thereof, and may be used in combination with other active ingredients such as commonly used detergents or additives such as dyes, surfactants or preservatives. The detergent may be produced as powder, granules, tablets or liquid according to the intended use thereof, and a conventional method may be used for packaging for commercialization thereof.

In addition, the content of the antimicrobial composition in the detergent may be appropriately selected depending on the intended use, application form, application site and application target article thereof, and may be, for example, 0.01 parts by weight to 50 parts by weight based on the total weight of the detergent composition, but the content of the antimicrobial composition is not limited thereto. Since the antimicrobial composition of the present disclosure is a natural component that is not harmful to the human body, it may be used in various amounts as long as it can achieve the application purpose due to the nature of the product.

In a preferred embodiment, the antimicrobial composition of the present disclosure may be applied as a cleanser.

The cleanser may be used as a hand, oral or feminine cleanser in a simple way in order to prevent pathogenic microorganism infection that often occurs in daily life. Since the antibacterial composition is a natural component that is not harmful to the human body, it can be safely applied as a hand, oral or feminine cleanser.

The cleanser may further contain additional additives selected appropriately according to the intended use thereof, and may be used in combination with other active ingredients or commonly used additives such as pigments, surfactants or preservatives. The cleaner may be produced as powder, granules, tablets or liquid according to the intended use thereof, but when it is produced in a liquid form, convenient use thereof will be possible.

In addition, the content of the antimicrobial composition in the cleanser may be appropriately selected depending on the intended use, application form, application site and application target article thereof, and may be, for example, 0.01 parts by weight to 50 parts by weight based on the total weight of the detergent composition, but the content of the antimicrobial composition is not limited thereto. Since the antimicrobial composition of the present disclosure is a natural component that is not harmful to the human body, it may be used in various amounts as long as it can achieve the application purpose due to the nature of the product.

Hereinafter, the present disclosure will be described in more detail with reference to specific examples. The following examples merely describe one preferred embodiment of the present disclosure, and the scope of the present disclosure is not limited by the contents described in the following examples.

### EXAMPLES

### Example 1: Wasp Collection and Wasp Extract Preparation

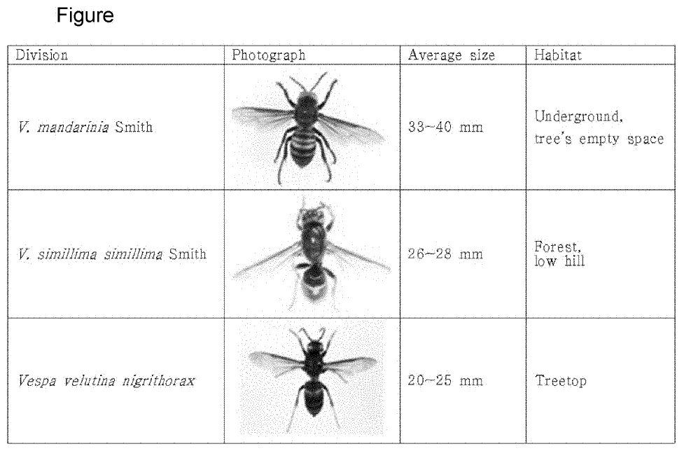

In August 2019, V. mandarinia Smith, V. simillima simillima Smith and Vespa velutina nigrithorax were collected in Gyeongsangbuk-do, Korea, and each wasp species was confirmed by an insect expert, Andong University professor Jeong Cheol-Hee (FIG. 1). The collected wasps were soaked in 30% ethanol without separate pretreatment and extracted therein for about 1 year. In this case, about 5 liters of 30% ethanol was added to 100 wasps. Thereafter, the wasps were removed, and the wasp extracts were filtered, and dried under reduced pressure and recovered.

### Example 2: Analysis of Components of Wasp Extracts

The total polyphenol, total flavonoid, total sugar and reducing sugar contents in each of the wasp extracts prepared in Example 1 were measured. For measurement of the total polyphenol content, 50 μl of Folin-ciocalteu and 100 μl of Na₂CO₃ saturated solution were added to 400 μl of each extract sample and allowed to stand at room temperature for 1 hour, and then the absorbance at 725 nm was measured. As a standard reagent, tannic acid was used. For measurement of the total flavonoid content, each extract sample was extracted by stirring with methanol for 18 hours and filtered, and 4 ml of 90% diethylene glycol was added to each of the filtrate samples and 40 μl of 1N NaOH was added thereto, and each of the mixtures was allowed to react at 37° C. for 1 hour, and then the absorbance at 420 nm was measured. As a standard reagent, rutin was used. Reducing sugar was quantified using a DNS method, and total sugar was quantified using a phenol-sulfuric acid method.

TABLE 1
Analysis of components of wasp extracts

| | Content (mg/g) | | | |
|---|---|---|---|---|
| | Total | Total | Total | Reducing |
| Division | polyphenol | flavonoid | sugar | sugar |
| V. mandarinia | 33.5 ± 0.9 | 3.5 ± 0.1 | 83.0 ± 6.3 | 54.8 ± 0.0 |
| Smith | | | | |
| Vespa velutina | 40.1 ± 0.1 | 8.0 ± 2.5 | 115.2 ± 4.8 | 60.6 ± 11.5 |
| nigrithorax | | | | |
| V. simillima | 29.8 ± 3.8 | 4.2 ± 0.4 | 121.2 ± 3.3 | 53.6 ± 1.6 |
| simillima Smith | | | | |

As a result, as shown in Table 1 above, the extracts of the three wasp species showed a total polyphenol content of 29.8 to 40.1 mg/g, and the Vespa velutina nigrithorax extract showed the highest total polyphenol content. The total flavonoid content was 3.5 to 8.0 mg/g and was the highest in the Vespa velutina nigrithorax extract. However, the total sugar content was the highest in the V. simillima simillima Smith extract (121.1 mg/g), which was 1.46 times higher than that in the V. mandarinia Smith extract, and the reducing sugar content was 53.6 to 60.6 mg/g and did not significantly differ between the extracts.

### Example 3: Evaluation of Antimicrobial Activities of Wasp Extracts

The antimicrobial activities prepared in Example were evaluated, and the results of the evaluation are shown in Table 2 below. For evaluation of the antibacterial activities, the following strains were used: Escherichia coli KCTC 1682, Proteus vulgaris KCTC 2433, Pseudomonas aeruginosa KACC 10186, and Salmonella typhimurium KCTC 1926, which are Gram-negative bacteria, and Staphylococcus aureus KCTC 1916, Staphylococcus epidermidis ATCC 12228, and Listeria monocytogenes KACC 10550, Bacillus subtilis KCTC 1924, which are Gram-positive bacteria. Meanwhile, for evaluation of the antifungal activities, Saccharomyces cerevisiae IF0 0233 and Candida albicans KCTC 1940 that causes Candidiasis fungal infection were used.

First, for evaluation of the antibacterial activities, each bacterial strain was inoculated into nutrient broth (Difco Co., USA) and cultured at 37° C. for 24 hours. Then, each strain was adjusted to an O.D.₆₀₀ of 0.1, and then 100 μl of each strain was plated on a Petri dish (90 mm, Green Cross, Korea) containing nutrient agar (Difco Co., USA) medium, and 5 μl of each extract sample added to sterile disc-paper (6.5 mm diameter, Whatsman No. 2) was added to each strain which was then incubated at 37° C. for 24 hours. For evaluation of the antifungal activities, each fungal strain was cultured using Sabouraud dextrose (Difco Co. USA) at 30° C. for 24 hours in the same manner as described above, and the antifungal activity of each extract sample was evaluated by measuring the size of the clear zone. As a control, each of the antibacterial agent ampicillin and the antifungal agent miconazole (Sigma Co., USA) was at a concentration of 1 μg/disc. The size of the clear zone was measured by measuring, in units of mm, the diameter of the part where growth was invisible with the naked eye, and was expressed as a representative result after three or more evaluations.

TABLE 2
Antimicrobial Activities of wasp extracts

| | | Clear zone (mm) | | | | |
|---|---|---|---|---|---|---|
| | | | | Wasp extract (500 μg/disc) | | |
| | | | | V. | Vespa | V. simillima |
| | | Antibiotic (1 μg/disc) | | mandarinia | velutina | simillima |
| Microorganisms | | Ampicillin | Miconazole | Smith | nigrithorax | Smith |
| Gram-negative | EC | 9.0 | — | — | — | — |
| bacteria | PA | 16.0 | — | 7.0 | 7.0 | 7.0 |
| | PV | 30.0 | — | 7.0 | 7.0 | 9.0 |
| | ST | 20.0 | — | 7.0 | 7.0 | — |
| Gram-positive | LM | 26.0 | — | 7.0 | 7.0 | — |
| bacteria | SE | 20.0 | — | — | — | — |
| | SA | 26.0 | — | — | — | — |
| | BS | 23.5 | — | 8.0 | 7.0 | 7.0 |
| Fungi | SC | — | 8.5 | — | — | — |
| | CA | — | 8.5 | — | — | — |

EC: Escherichia coli,
PA: Pseudomonas aeruginosa,
PV: Proteus vulgaris,
ST: Salmonella typhimurium,
LM: Listeria monocytogenes,
SE: Staphylococcus epidermidis,
SA: Staphylococcus aureus,
BS: Bacillus subtilis,
SC: Saccharomyces cerevisiae,
CA: Candida albicans,
—: no clear zone.

As shown in Table 2 above, ampicillin (1 μg/disc) used as an antibacterial activity control exhibited a clear zone size of 9.0 to 30.0 mm for the gram-positive bacteria and the gram-negative bacteria, suggesting that ampicillin exhibits broad-spectrum and strong antibacterial activity. The antifungal agent miconazole (1 μg/disc) exhibited a clear zone size of 8.5 mm for each of C. albicans and S. cerevisiae. Meanwhile, the V. mandarinia Smith, Vespa velutina nigrithorax or V. simillima simillima Smith extract exhibited weak antibacterial activity against the gram-negative bacteria Proteus vulgaris and Pseudomonas aeruginosa at a concentration of 500 μg/disc, and showed antibacterial activity against the gram-positive bacterium Bacillus subtilis. However, the extract did not exhibit strong antibacterial activity, unlike the existing antibiotics.

### Example 4: Preparation of Sequential Organic Solvent Fractions of Each of V. mandarinia Smith, Vespa velutina nigrithorax or V. simillima simillima Smith Extracts and Analysis of Components of the Fractions

Through Example 3, it was confirmed that the wasp extracts had excellent antibacterial activity. Thus, the wasp extracts were fractionated sequentially with organic solvents (hexene, ethyl acetate and butanol), and water residue was finally recovered. Table 3 below shows the results of analyzing organic solvent fractionation efficiency and the components of the fractions.

TABLE 31
Preparation of sequential organic solvent
fractions of each of V. mandarinia Smith, Vespa velutina
nigrithorax and V. simillima simillima Smith extracts and
analysis of components of the fractions

| | | | Content (mg/g) | | | |
|---|---|---|---|---|---|---|
| | | Fractionation | Total | Total | Total | Reducing |
| Division | | yield (%) | polyphenol | flavonoid | sugar | sugar |
| Vespa | Hexene | 0.9 | 22.9 ± 0.7 | 7.5 ± 0.1 | 35.7 ± 0.6 | 5.6 ± 0.1 |
| velutina | fraction | | | | | |
| nigrithorax | Ethyl | 12.5 | 26.0 ± 1.6 | 12.0 ± 0.1 | 136.3 ± 11.2 | 6.9 ± 0.6 |
| extract | acetate | | | | | |
| | fraction | | | | | |
| | Butanol | 53.2 | 54.1 ± 2.7 | 4.5 ± 0.6 | 116.4 ± 0.1 | 13.8 ± 2.4 |
| | fraction | | | | | |
| | Water | 33.4 | 40.8 ± 1.6 | 4.0 ± 0.3 | 105.1 ± 2.1 | 20.4 ± 0.7 |
| | residue | | | | | |
| V simillima | Hexene | 1.8 | 20.6 ± 0.2 | 5.7 ± 0.1 | 27.8 ± 0.1 | 1.2 ± 0.0 |
| simillima | fraction | | | | | |
| Smith extract | Ethyl | 5.3 | 13.2 ± 0.2 | 9.7 ± 1.0 | 115.6 ± 3.1 | 6.1 ± 0.2 |
| | acetate | | | | | |
| | fraction | | | | | |
| | Butanol | 81.2 | 50.1 ± 1.8 | 2.9 ± 0.0 | 150.7 ± 2.7 | 14.0 ± 0.0 |
| | fraction | | | | | |
| | Water | 11.7 | 41.8 ± 0.5 | 2.8 ± 0.1 | 74.3 ± 5.7 | 16.2 ± 0.2 |
| | residue | | | | | |
| V. mandarinia | Hexene | 0.2 | ND | ND | ND | ND |
| Smith extract | fraction | | | | | |
| | Ethyl | 6.1 | 39.3 ± 0.4 | 7.3 ± 0.5 | 102.3 ± 3.1 | 5.9 ± 0.4 |
| | acetate | | | | | |
| | fraction | | | | | |
| | Butanol | 85.8 | 26.8 ± 1.0 | 2.5 ± 0.0 | 76.6 ± 1.3 | 12.9 ± 0.4 |
| | fraction | | | | | |
| | Water | 7.9 | 13.8 ± 0.4 | 2.5 ± 0.1 | 30.4 ± 2.2 | 5.8 ± 0.4 |
| | residue | | | | | |

As shown in Table 3 above, it could be seen that 53.24% of the Vespa velutina nigrithorax extract was fractionated into the butanol fraction and 33.4% of the extract was recovered as water residue, suggesting that most of the extract was a water-soluble component. In addition, the ethyl acetate fraction of the extract accounted for 12.5%. As a result of measuring the total polyphenol content of each fraction, the total polyphenol content was the highest in the butanol fraction (54.1 mg/g), was 40.8 mg/g in the water residue, and was relatively low in the ethyl acetate fraction (26.0 mg/g). However, as a result of analyzing the total flavonoid content, the total flavonoid content was the highest in the ethyl acetate fraction (12.0 mg/g). The total sugar content was the highest in the ethyl acetate fraction (136.3 mg/g), and the reducing sugar content was the highest in the water residue (20.4 mg/g). Therefore, it was confirmed that the ethyl acetate fraction contained large amounts of compounds having polyphenol/flavonoid structures to which saccharides are bound.

Meanwhile, it could be seen that 81.2% of the V. simillima simillima Smith extract was fractionated into the butanol fraction and 11.7% of the extract was recovered as water residue, suggesting that most of the extract was a water-soluble component, similar to the Vespa velutina nigrithorax extract. In addition, the ethyl acetate fraction of the extract accounted for 5.3%. As a result of measuring the total polyphenol content of each fraction, the total polyphenol content was the highest in the butanol fraction (50.1 mg/g), was 41.8 mg/g in the water residue, and was relatively low in the ethyl acetate fraction (13.2 mg/g). However, as a result of analyzing the total flavonoid content, the total flavonoid content was the highest in the ethyl acetate fraction (9.7 mg/g). The total sugar content was the highest in the butanol fraction (150.7 mg/g), whereas the reducing sugar content was the highest in the water residue (16.2 mg/g). Therefore, it was confirmed that the ethyl acetate fraction contained large amounts of compounds having polyphenol/flavonoid structures to which saccharides are bound.

Finally, it could be seen that 85.8% of the V. mandarinia Smith extract was fractionated into the butanol fraction and 7.9% of the extract was recovered as water residue, suggesting that most of the extract was a water-soluble component, similar to the Vespa velutina nigrithorax extract. In addition, the ethyl acetate fraction of the extract accounted for 6.1%. As a result of measuring the total polyphenol content of each fraction, the total polyphenol content was the highest in the ethyl acetate fraction (39.3 mg/g), was 26.8 mg/g in the butanol fraction, and was relatively low in the water residue (13.8 mg/g). However, as a result of analyzing the total flavonoid content, the total flavonoid content was the highest in the ethyl acetate fraction (7.3 mg/g). The total sugar content was unusually the highest in the ethyl acetate fraction (102.3 mg/g), whereas the reducing sugar content was the highest in the butanol fraction (12.9 mg/g). Therefore, it was confirmed that the ethyl acetate fraction contained large amounts of compounds having polyphenol/flavonoid structures to which saccharides are bound.

### Example 5: Evaluation of Antimicrobial Activities of Fractions of Each of Vespa velutina Nigrithorax, V. Simillima Simillima Smith and V. mandarinia Smith Extracts

The antimicrobial activities of the fraction of each of the Vespa velutina nigrithorax, V. simillima simillima Smith and V. mandarinia Smith extracts, prepared in Example 4, were measured according to the same method as the antibacterial and antifungal activity evaluation described in Example 3. The results of the evaluation are shown in Tables 4 to 6 below.

TABLE 4
Evaluation of antimicrobial activities of
fractions of Vespa velutina nigrithorax ethanol extract

| | | Clear zone (mm) | | | | | | |
|---|---|---|---|---|---|---|---|---|
| | | | | Vespa velutina nigrithorax (500 μg/disc) | | | | |
| | | | | | | Ethyl | | |
| | | Antibiotic (1 μg/disc) | | Ethanol | Hexane | acetate | Butanol | Water |
| Microorganisms | | Ampicillin | Miconazole | extract | fraction | fraction | fraction | residue |
| Gram- | EC | 9.0 | — | — | NA | 10.0 | — | — |
| negative | PA | 16.0 | — | 7.0 | NA | 10.0 | — | — |
| bacteria | PV | 30.0 | — | 7.0 | NA | 14.0 | — | — |
| | ST | 20.0 | — | 7.0 | NA | 7.0 | — | — |
| Gram- | LM | 26.0 | — | 7.0 | NA | 13.0 | — | — |
| positive | SE | 20.0 | — | — | NA | 8.5 | — | — |
| bacteria | SA | 26.0 | — | — | NA | 9.0 | — | — |
| | BS | 23.5 | — | 8.0 | NA | 10.0 | — | — |
| Fungi | SC | — | 8.5 | — | NA | 7.5 | 7.0 | 7.0 |
| | CA | — | 8.5 | — | NA | 8.0 | 7.0 | 7.0 |

TABLE 5
Evaluation of antimicrobial activities of
fractions of V. simillima simillima Smith ethanol extract

| | | Clear zone (mm) | | | | | | |
|---|---|---|---|---|---|---|---|---|
| | | | | V. simillima simillima Smith (500 μg/disc) | | | | |
| | | | | | | Ethyl | | |
| | | Antibiotic (1 μg/disc) | | Ethanol | Hexene | acetate | Butanol | Water |
| Microorganisms | | Ampicillin | Miconazole | extract | fraction | fraction | fraction | residue |
| Gram- | EC | 9.0 | — | — | — | 8.0 | — | — |
| negative | PA | 16.0 | — | 7.0 | — | 9.5 | — | — |
| bacteria | PV | 30.0 | — | 7.0 | 9.0 | 9.0 | — | — |
| | ST | 20.0 | — | 7.0 | = | 8.0 | — | — |
| Gram- | LM | 26.0 | — | 7.0 | — | 11.0 | — | — |
| positive | SE | 20.0 | — | — | 7.0 | 8.5 | — | — |
| bacteria | SA | 26.0 | — | — | 8.0 | 7.5 | — | — |
| | BS | 23.5 | — | 8.0 | 8.0 | 9.0 | 8.5 | — |
| Fungi | SC | — | 8.5 | — | 7.5 | 7.5 | 7.0 | 7.0 |
| | CA | — | 8.5 | — | 8.0 | 8.0 | 7.0 | 7.0 |

TABLE 6
Evaluation of antimicrobial activities of
fractions of V. mandarinia Smith ethanol extract

| | | Clear zone (mm) | | | | | | |
|---|---|---|---|---|---|---|---|---|
| | | | | V. mandarinia Smith (500 μg/disc) | | | | |
| | | | | | | Ethyl | | |
| | | Antibiotic (1 μg/disc) | | Ethanol | Hexene | acetate | Butanol | Water |
| Microorganisms | | Ampicillin | Miconazole | extract | fraction | fraction | fraction | residue |
| Gram- | EC | 9.0 | — | — | NA | — | 8.0 | — |
| negative | PA | 16.0 | — | 7.0 | NA | — | 7.5 | — |
| bacteria | PV | 30.0 | — | 7.0 | NA | — | 7.5 | — |
| | ST | 20.0 | — | 7.0 | NA | — | 7.0 | — |
| Gram- | LM | 26.0 | — | 7.0 | NA | — | 7.0 | — |
| positive | SE | 20.0 | — | — | NA | — | — | — |
| bacteria | SA | 26.0 | — | — | NA | — | — | — |
| | BS | 23.5 | — | 8.0 | NA | 9.5 | — | — |
| Fungi | SC | — | 8.5 | — | NA | — | — | — |
| | CA | — | 8.5 | — | NA | — | — | — |

As a result, it was confirmed that the ethyl acetate fraction of each of the Vespa velutina nigrithorax extract and the V. simillima simillima Smith extract exhibited strong antimicrobial activity and also exhibited antimicrobial activity against all the gram-positive bacteria, the gram-negative bacteria and the fungi. However, it was confirmed that the ethyl acetate fraction of the V. mandarinia Smith extract exhibited antibacterial activity against only Bacillus subtilis, and the butanol fraction thereof exhibited antibacterial activity against the gram-negative bacteria. The above results suggest that the ethyl acetate fraction of each of the Vespa velutina nigrithorax extract and the V. simillima simillima Smith extract can be effectively used for the control of food poisoning-causing bacteria and pathogenic bacteria.

### Example 6: Evaluation of Erythrocyte Hemolytic Activities of Wasp Extracts and Fractions Thereof

In order to evaluate the acute toxicities of the Vespa velutina nigrithorax, V. simillima simillima Smith and V. mandarinia Smith extracts and the fractions thereof, the human erythrocyte hemolytic activities thereof were evaluated. The results of the evaluation are shown in Table 7 below. At this time, the hemolytic activity was evaluated according to a previous report (Son Ho-Yong et al., 2014, Korean J. Microbiol. Biotechnol. 42: 285-292). Briefly, 100 μl of human erythrocytes were washed three times with PBS and added to a 96-well microplate, and 100 μl of a sample solution at various concentrations was added thereto and then allowed to react at 37° C. for 30 minutes. Thereafter, the reaction solution was centrifuged at 1,500 rpm for 10 minutes, and 100 μl of the supernatant was transferred to a fresh microtiter plate, and then the degree of hemoglobin leakage following hemolysis was measured at 414 nm. DMSO (2%) was used as a solvent control for the sample, and Triton X-100 (1 mg/ml) was used as an experimental control for hemolysis of erythrocytes. Hemolytic activity was calculated using the following equation.

(%) Hemolysis=[(Abs. S−Abs. C)/(Abs. T−Abs. C)]×100

Abs. S: absorbance of sample-added group

Abs. C: absorbance of DMSO-added group

Abs. T: absorbance of Triton X-100-added group

TABLE 7
Evaluation of human erythrocyte hemolytic activities of
Vespa velutina nigrithorax, V. simillima simillima Smith
and V. mandarinia Smith extracts and fractions thereof

| | | Concentration | Human erythrocyte |
|---|---|---|---|
| Sample/control | | (mg/ml) | hemolytic activity |
| Distilled water | | — | 0.0 ± 0.3 |
| Solvent (DMSO) | | — | 1.5 ± 1.0 |
| Triton X-100 | | 1.0 | 100.0 ± 0.1 |
| Amphotericin B | | 0.1 | 95.8 ± 0.3 |
| | | 0.05 | 72.4 ± 5.2 |
| | | 0.025 | 59.5 ± 2 |
| | | 0.0125 | 48.2 ± 5.9 |
| | | 0.0063 | 21.6 ± 3.7 |
| | | 0.0032 | 6.5 ± 0.9 |
| | | 0 | 0.0 ± 1.7 |
| Vespa velutina | Ethanol extract | 1.0 | 0.7 ± 0.7 |
| nigrithorax | Hexene fraction | 1.0 | 2.0 ± 0.5 |
| | Ethyl acetate | 1.0 | −2.5 ± 0.2 |
| | fraction | | |
| | Butanol fraction | 1.0 | 0.1 ± 0.6 |
| | Water residue | 1.0 | 1.1 ± 0.1 |
| V. simillima | Ethanol extract | 1.0 | −11.2 ± 0.2 |
| simillima Smith | Hexene fraction | 1.0 | NA |
| | Ethyl acetate | 1.0 | 8.8 ± 1.9 |
| | fraction | | |
| | Butanol fraction | 1.0 | −1.5 ± 0.3 |
| | Water residue | 1.0 | 0.6 ± 2.7 |
| V. mandarinia | Ethanol extract | 1.0 | −7.1 ± 0.5 |
| Smith | Hexene fraction | 1.0 | NA |
| | Ethyl acetate | 1.0 | −4.7 ± 1.7 |
| | fraction | | |
| | Butanol fraction | 1.0 | −2.4 ± 0.6 |
| | Water residue | 1.0 | −2.2 ± 0.8 |

As shown in Table 7 above, it was confirmed that DMSO and water used as controls had no hemolytic activity, and Triton X-100 hemolyzed 100% of erythrocytes at a concentration of 1 mg/ml. Amphotericin B, which is used as an anticancer agent, hemolyzed 59% or more of erythrocytes at a concentration of 0.025 mg/ml. Meanwhile, the Vespa velutina nigrithorax and V. mandarinia Smith extracts and the fractions thereof showed no erythrocyte hemolytic activity even at a concentration of 1.0 mg/ml, and the fractions (excluding ethyl acetate fraction) of the V. simillima simillima Smith extract also showed no erythrocyte hemolytic activity. The ethyl acetate fraction of the V. simillima simillima Smith extract showed an erythrocyte hemolytic activity of about 8.8%, which was not toxic. Thus, it is expected that the ethyl acetate fraction of each of the Vespa velutina nigrithorax and V. simillima simillima Smith extracts will show no separate acute toxicity.

### Example 7: Evaluation of Plasma, Acid and Heat Stabilities of Ethyl Acetate Fraction of Each of Vespa velutina nigrithorax and V. simillima simillima Smith Extracts

The heat stability and acid stability for antithrombotic activity of the ethyl acetate fraction of each of the Vespa velutina nigrithorax and V. simillima simillima Smith extracts, obtained in Example 4, were evaluated. The fraction maintained excellent activity without significant loss of its antibacterial and antifungal activities even when it was heat-treated at 100° C. for 1 hour or treated at pH 2 (0.01 M HCl) for 1 hour. Therefore, it is expected that the ethyl acetate fraction of each of the Vespa velutina nigrithorax and V. simillima simillima Smith extracts will maintain excellent antimicrobial activity even in various food, cosmetic and pharmaceutical processing processes.

As demonstrated through the Examples of the present disclosure, the Vespa velutina nigrithorax extract or the V. simillima simillima Smith extract as an active ingredient for the antimicrobial composition of the present disclosure has an excellent effect in that it exhibits excellent broad-spectrum antimicrobial activity against gram-positive bacteria, gram-negative bacterial and fungi, and thus may be used for pharmaceutical compositions, health functional foods, cosmetic compositions, animal feed compositions, additives for food, cosmetics or animal feed, various detergents and various cleansers. In addition, the Vespa velutina nigrithorax extract or V. simillima simillima Smith extract of the present disclosure has excellent heat stability, and does not lose its activity even under an acidic condition of pH 2. Accordingly, the extract may be easily processed into various forms such as liquid, cream, powder, pills or tablets, and thus is very useful in the pharmaceutical, food, livestock, health care and cosmetic industries.

What is claimed is:

1. An antimicrobial composition containing, as an active ingredient, a wasp extract selected from the group consisting of a Vespa velutina nigrithorax extract and a V. simillima simillima Smith extract.

2. The antimicrobial composition of claim 1, wherein the extract is an ethanol extract.

3. The antimicrobial composition of claim 1, wherein the extract is an ethyl acetate fraction obtained after ethanol extraction.

4. The antimicrobial composition of claim 1, which is selected from the group consisting of pharmaceutical compositions, health functional foods, cosmetic compositions, animal feed compositions, food additives, cosmetic additives, animal feed additives, detergents, and cleansers.
